(19) United States
(12) Patent Application Publication
(10) Pub. No.: US 2018/0092606 A1
(43) Pub. Date: Apr. 5, 2018

(54) Heart Sound Processing Method and System for Detecting Cardiopathy

(71) Applicant: National Kaohsiung University of Applied Sciences, Kaohsiung (TW)

(72) Inventor: Gwo-Jia Jong, Kaohsiung (TW)

(21) Appl. No.: 15/284,599

(22) Filed: Oct. 4, 2016

(51) Int. Cl. A61B 5/00; A61B 7/02; A61B 5/02; A61B 5/0402

(52) U.S. Cl. CPC A61B 5/7282; A61B 5/7257; A61B 5/726; A61B 5/0402; A61B 5/7246; A61B 5/02028; A61B 7/02

(57) ABSTRACT

A heart sound processing method for detecting cardiopathy includes: partitioning heart sound data to obtain a plurality of heart sound data fragments; converting the heart sound data fragments with continuous wavelet transformation to obtain CWT (continuous wavelet transformation) data; converting the heart sound data fragments with short-time Fourier transformation to obtain STFT (short-time Fourier transformation) data; and comparing the CWT data and the STFT data with at least one ultrasound data sample of cardiopathy to seek at least one correlation between time and frequency for identifying cardiopathy.

### BACKGROUND OF THE INVENTION

### 1. Field of the Invention

The present invention relates to a heart sound processing method and system for detecting cardiopathy. Particularly, the present invention relates to the heart sound processing method and system comparing with ultrasound data for precisely detecting cardiopathy.

### 2. Description of the Related Art

U.S. Patent Application Publication No. 20120289848, entitled “Method and System for Discriminating Heart Sound and Cardiopathy,” discloses a method, including processing a specific function calculation on heart-sound signals to generate a first calculation signal and suppressing noises of the heart-sound signal; transforming the filtering signal to generate data for an image plots; comparing the image plot with data of heart-sound plots to obtain a comparison result for discriminating the heart sound.

The system for discriminating heart sound from cardiopathy includes a signal receiving unit, a signal processing unit, a storage unit, an output unit and a display unit. The signal receiving unit is provided for receiving a heart-sound signal “A”. The signal processing unit further includes a first calculation unit, a filter unit, a second calculation unit and a comparison unit. The first calculation unit has a specific function calculation on the heart-sound signal “A” to generate a first calculation signal “X”, with the specific function calculation based on the product of the natural log of the absolute value of the heart-sound signal “A” multiplied by the heart-sound signal “A”, such as X=cAln|A′| with c being any value or function value, with A′=A if A≠0 and A′=R if A=0 (R≧1 and R is a real number).

The filter unit is provided to filter the first calculation signal “X” to generate a filtering signal “Y”. The second calculation unit is provided to calculate the filtering signal “Y” to generate a plurality of intrinsic mode function (IMF) bands and data “Z” corresponding to an image plot according to at least one of the required IMF bands. For example, the image plot is a time-frequency plot.

In addition, the storage unit is provided to store heart-sound-plot data and is further provided with a cardiopathy heart-sound-plot database. The output unit is a wireless transmission module or a wired transmission interface. The comparison unit is provided to compare the image plot with the heart-sound-plot data to generate a comparison result “CR” which is further transmitted to the display unit via the output unit for discriminating heart sound.

However, the above method requires generating data “Z” corresponding to the image plot and comparing image plot with the heart-sound-plot data which results in complicating the entire process. Hence, there is a need of improving the conventional heart sound plotting method for discriminating cardiopathy from heart sound. The above-mentioned patent is incorporated herein by reference for purposes including, but not limited to, indicating the background of the present invention and illustrating the situation of the art.

As is described in greater detail below, the present invention provides a heart sound processing method and system for detecting cardiopathy. A heart sound data is partitioned to obtain a plurality of heart sound data fragments which are converted with continuous wavelet transformation (CWT) into CWT data and are further converted with short-time Fourier transformation (STFT) into STFT data. The CWT data and the STFT data are pre-compared with ultrasound data samples of cardiopathy to seek at least one correlation between time and frequency for identifying cardiopathy. Advantageously, the present invention can rapidly and precisely identify cardiopathy from heart sound in such a way as to mitigate and overcome the above-mentioned problem of the conventional heart sound plotting method.

### SUMMARY OF THE INVENTION

The primary objective of this invention is to provide a heart sound processing method and system for detecting cardiopathy. A heart sound data is partitioned to obtain a plurality of heart sound data fragments which are converted with continuous wavelet transformation (CWT) into CWT data and are further converted with short-time Fourier transformation (STFT) into STFT data. The CWT data and the STFT data are pre-compared with ultrasound data samples of cardiopathy to seek at least one correlation between time and frequency for identifying cardiopathy. Advantageously, the heart sound processing method and system of the present invention is successful in rapidly and precisely identifying cardiopathy from heart sound. The heart sound processing method for detecting cardiopathy in accordance with an aspect of the present invention includes:

partitioning first heart sound data to obtain a plurality of first heart sound data fragments;

converting the first heart sound data fragments with continuous wavelet transformation to obtain first CWT data;

converting the first heart sound data fragments with short-time Fourier transformation to obtain first STFT data; and

calculating the first CWT data or the first STFT data to seek at least one correlation between time and frequency for identifying cardiopathy.

The heart sound processing method for detecting cardiopathy in accordance with a separate aspect of the present invention includes:

partitioning first heart sound data to obtain a plurality of first heart sound data fragments;

converting the first heart sound data fragments with continuous wavelet transformation to obtain first CWT data;

converting the first heart sound data fragments with short-time Fourier transformation to obtain first STFT data; and

comparing the first CWT data and the first STFT data with at least one ultrasound data sample of cardiopathy to seek at least one correlation between time and frequency for identifying cardiopathy.

In a separate aspect of the present invention, the at least one correlation is applied to identify cardiopathy from second CWT data and second STFT data of second heart sound data collected from another patient to generate a predictable result of correlation coefficients.

In a further separate aspect of the present invention, the predictable result of correlation coefficients includes a disease of ventricular septal defect or atrial septal defect.

In yet a further separate aspect of the present invention, the first CWT data and the first STFT data are compared with the ultrasound data to seek a maximum frequency point of heart sound, a maximum amplitude point of heart sound and at least one time interval of two maximum frequency points or two maximum amplitude points.

In yet a further separate aspect of the present invention, the first CWT data and the first STFT data are calculated with Pearson product-moment coefficient.

In yet a further separate aspect of the present invention, the first heart sound data is compared with ECG data for identifying cardiopathy.

The heart sound processing system for detecting cardiopathy in accordance with an aspect of the present invention includes:

a heart sound receiving unit provided to receive heart sound data;

a heart sound processing unit connected with the heart sound receiving unit, with partitioning the heart sound data to obtain a plurality of heart sound data fragments, with converting the heart sound data fragments with continuous wavelet transformation to obtain CWT data, with converting the heart sound data fragments with short-time Fourier transformation to obtain STFT data;

a data storage unit connected with the heart sound processing unit, with the data storage unit storing at least one set of ultrasound data samples of cardiopathy; and

an output unit connected with the heart sound processing unit, with the output unit outputting a predictable result of correlation coefficients;

wherein the first CWT data and the first STFT data are compared with the at least one set of ultrasound data samples of cardiopathy and are further calculated to seek at least one correlation between time and frequency for identifying cardiopathy.

In a separate aspect of the present invention, the heart sound receiving unit includes a first receiver unit and a second receiver unit to attach to a first predetermined position and a second predetermined position for synchronously collecting different heart sound data.

In a further separate aspect of the present invention, the heart sound receiving unit is configured to attach to a predetermined position of human skin.

In yet a further separate aspect of the present invention, heart sounds of the heart sound data fragments have a range of frequencies between 1 Hz and 100 Hz.

In yet a further separate aspect of the present invention, each of the heart sound data fragments includes a predetermined amount of continuous heart sound signals.

In yet a further separate aspect of the present invention, a pathologic murmur signal is detected in the heart sound data fragment to identify cardiopathy.

In yet a further separate aspect of the present invention, the pathologic murmur signal includes a systolic heart murmur signal or a diastolic heart murmur signal.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a schematic view of a heart sound processing system in accordance with a preferred embodiment of the present invention.

FIG. 2 is a flowchart of a heart sound processing method for detecting cardiopathy in accordance with a preferred embodiment of the present invention.

FIG. 3A is two phonocardiographs of heart sound data applied in the heart sound processing method and system for detecting cardiopathy in accordance with the preferred embodiment of the present invention.

FIG. 3B is an electrocardiogram applied in the heart sound processing method and system for detecting cardiopathy in accordance with the preferred embodiment of the present invention.

FIG. 4A is a phonocardiograph of first heart sound data applied in the heart sound processing method and system for detecting cardiopathy in accordance with the preferred embodiment of the present invention.

FIG. 4B is a phonocardiograph of second heart sound data applied in the heart sound processing method and system for detecting cardiopathy in accordance with the preferred embodiment of the present invention.

FIG. 5A is an image of CWT data of slight aortic regurgitation and slight aortic stenosis collected from a first patient and processed by the heart sound processing method and system for detecting cardiopathy in accordance with the preferred embodiment of the present invention.

FIG. 5B is an image of CWT data of continuous murmur and patent ductus arteriosum collected from a second patient and processed by the heart sound processing method and system for detecting cardiopathy in accordance with the preferred embodiment of the present invention.

FIG. 6A is an image of STFT data of slight aortic regurgitation (AR) and slight aortic stenosis (AS) collected from the first patient and processed by the heart sound processing method and system for detecting cardiopathy in accordance with the preferred embodiment of the present invention.

FIG. 6B is an image of STFT data of continuous murmur and patent ductus arteriosum (PDA) collected from the second patient and processed by the heart sound processing method and system for detecting cardiopathy in accordance with the preferred embodiment of the present invention.

FIG. 7A is a series of images of first original heart sound data fragment collected from the first patient to compare with first CWT data and STFT data processed by the heart sound processing method and system for detecting cardiopathy in accordance with the preferred embodiment of the present invention.

FIG. 7B is a series of images of second original heart sound data fragment collected from to compare with second CWT data and STFT data the first patient processed by the heart sound processing method and system for detecting cardiopathy in accordance with the preferred embodiment of the present invention.

FIG. 8A is a series of images of first original heart sound data fragment collected from the second patient to compare with first CWT data and STFT data processed by the heart sound processing method and system for detecting cardiopathy in accordance with the preferred embodiment of the present invention.

FIG. 8B is a series of images of second original heart sound data fragment collected from the second patient to compare with second CWT data and STFT data processed by the heart sound processing method and system for detecting cardiopathy in accordance with the preferred embodiment of the present invention.

### DETAILED DESCRIPTION OF THE INVENTION

It is noted that a heart sound processing method and system thereof for detecting cardiopathy in accordance with the preferred embodiment of the present invention can be applicable to various cardiopathy detecting systems or related devices, including home care systems, medical care auto-control systems, telehealth systems or teaching hospital systems for example, which are not limitative of the present invention.

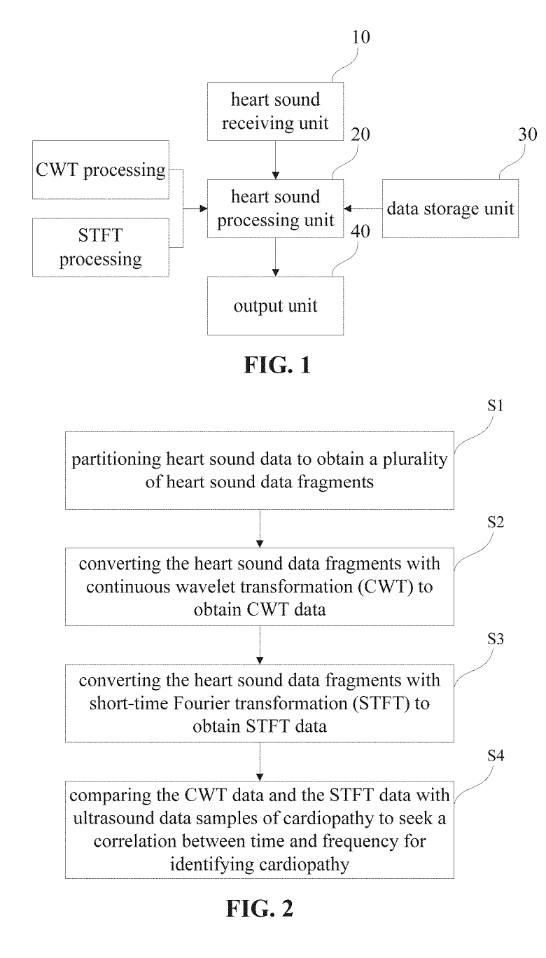

FIG. 1 shows a schematic view of a heart sound processing system in accordance with a preferred embodiment of the present invention. Referring now to FIG. 1, the heart sound processing system in accordance with the preferred embodiment of the present invention includes a heart sound receiving unit 10, a heart sound processing unit 20, a data storage unit 30 and an output unit 40. In a preferred embodiment, the heart sound receiving unit 10 includes a first receiver (patch) unit and a second receiver (patch) unit and is configured to attach to predetermined positions of human skin.

With continued reference to FIG. 1, by way of example, the data storage unit 30 to store ultrasound data samples or cardiopathy ultrasound data samples for comparing with heart sound data. Accordingly, the data storage unit 30 can be applied to build a database with a plurality of pattern samples of correlation between time and frequency for identifying cardiopathy.

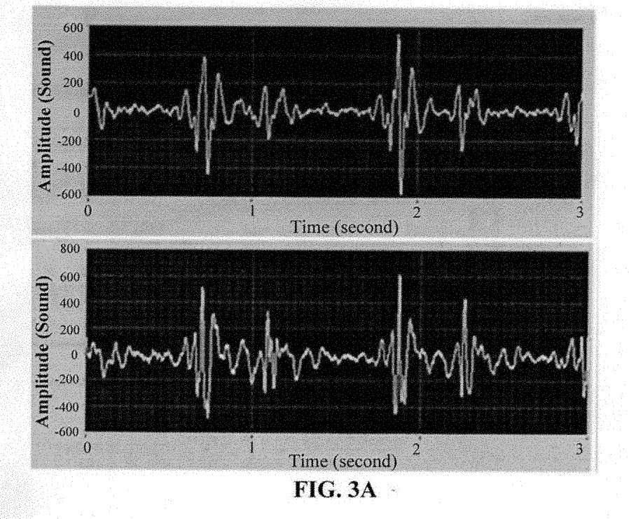

FIG. 2 shows a flowchart of a heart sound processing method for detecting cardiopathy in accordance with a preferred embodiment of the present invention. Referring now to FIGS. 1 and 2, the heart sound processing method in accordance with the preferred embodiment of the present invention includes the step S1: measuring heart sound data and other related data (e.g. ECG data, electrocardiogram data) by the heart sound receiving unit 10 or a heart sound measuring device of AUDICOR® products from U.S., and automatically or semi-automatically partitioning the heart sound data by the heart sound processing unit 20 to obtain a plurality of heart sound data fragments. In a preferred embodiment, the ECG data are also partitioned to form a plurality of corresponding ECG data fragments.

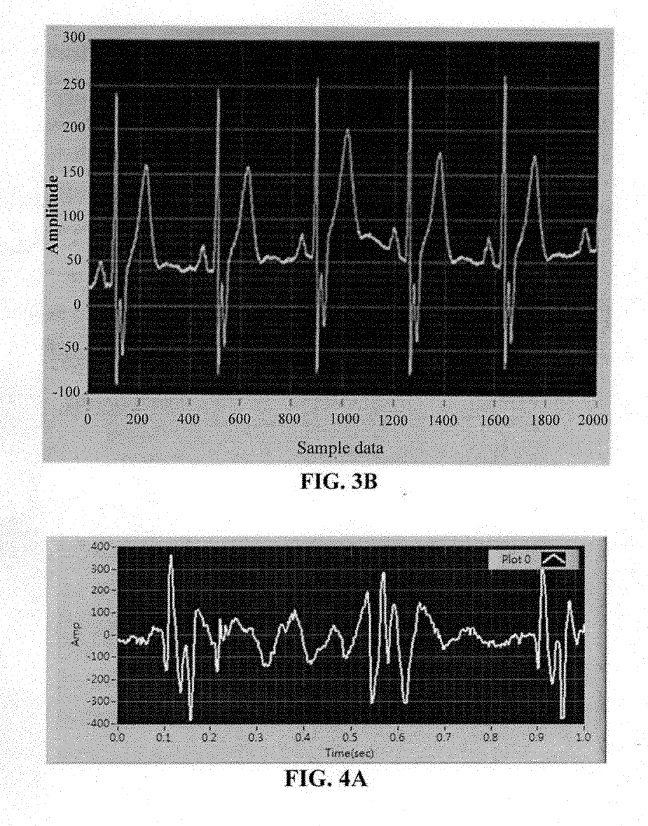

FIG. 3A shows two phonocardiographs (PCGs) of heart sound data applied in the heart sound processing method and system for detecting cardiopathy in accordance with the preferred embodiment of the present invention. FIG. 3B shows an electrocardiogram (ECG), corresponding to heart sound data shown in FIG. 3A, applied in the heart sound processing method and system for detecting cardiopathy in accordance with the preferred embodiment of the present invention. Referring to FIGS. 1, 2, 3A and 3B, the two separate heart sound data are synchronously measured by two separate heart sound measuring devices for detecting cardiopathy, with also synchronously measuring the ECG.

Generally, hear sounds include first hear sounds (so-called S1), second hear sounds (so-called S2), third hear sounds (so-called S3) and fourth hear sounds (so-called S4) which are caused by systolic actions of cardiac muscles, shutting of cardiac valves, blood flows in ventricles or vibrations of artery walls. Systolic and diastolic actions of cardiac muscles generate first hear sounds and “S1” and second hear sounds “S2” which can be easily measured by a stethoscope or an instrument. Actually, third hear sounds “S3” usually generate at childhood or early teenage and fourth hear sounds “S4” are rarely detected.

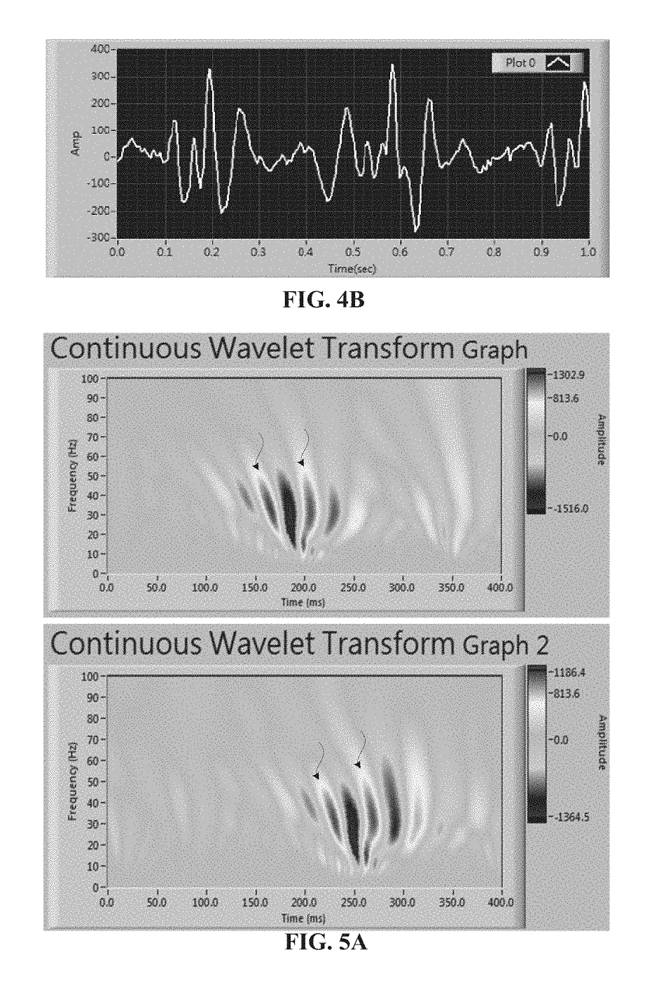

FIG. 4A shows a first phonocardiograph of first heart sound data (S1) applied in the heart sound processing method and system for detecting cardiopathy in accordance with the preferred embodiment of the present invention and FIG. 4B shows a second phonocardiograph of second heart sound data (S2) applied in the heart sound processing method and system for detecting cardiopathy in accordance with the preferred embodiment of the present invention. Referring to FIGS. 4A and 4B, the first heart sound data (S1) are collected at a first position of heart peripheral areas by a first patch while the second heart sound data (S2) are synchronously collected at a second position of heart peripheral areas by a second patch. By way of example, each of the heart sound data fragments includes a predetermined amount of continuous heart sound signals, 400 data points or other number of data points. In a preferred embodiment, the heart sounds of the heart sound data fragments have a range of frequencies between 1 Hz and 100 Hz or other low frequency range.

Referring again to FIGS. 1 and 2, the heart sound processing method in accordance with the preferred embodiment of the present invention includes the step S2: automatically or semi-automatically converting the heart sound data fragments with continuous wavelet transformation (CWT) by the heart sound processing unit 20 to obtain CWT data which can be shown as a first cardiopathy pattern.

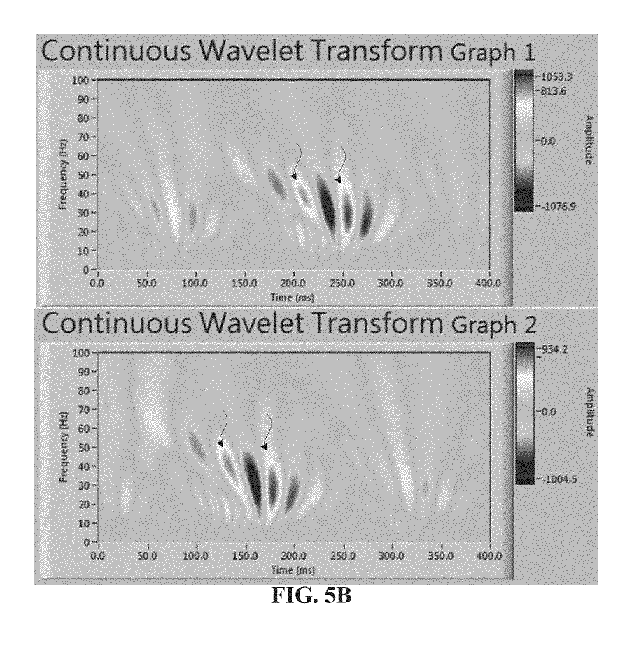

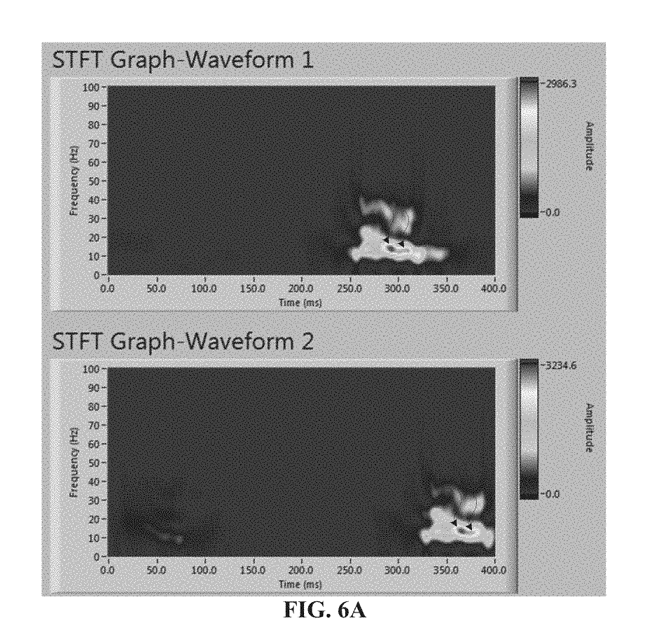

FIG. 5A shows an image of CWT data (two fragments) of slight aortic regurgitation and slight aortic stenosis collected from a first patient and processed by the heart sound processing method and system for detecting cardiopathy in accordance with the preferred embodiment of the present invention. Referring to FIG. 5A, after CWT processed, a first CWT pattern, as shown in the upper portion in FIG. 5A, and a second CWT pattern, as shown in the lower portion in FIG. 5A, are obtained to compare the CWT data. Apparently, a first pattern sample of the CWT data has a first maximum amplitude and a second maximum amplitude, as best shown in two arrows in FIG. 5A, which indicate the heart of first patient suffering from diseases of slight aortic regurgitation (AR) and slight aortic stenosis (AS).

Next, FIG. 5B further shows an image of CWT data (two fragments) of continuous murmur and patent ductus arteriosum collected from a second patient and processed by the heart sound processing method and system for detecting cardiopathy in accordance with the preferred embodiment of the present invention. Referring to FIG. 5B, after CWT processed, a first CWT pattern within a first main pattern, as shown in the upper portion in FIG. 5B, and a second CWT pattern within a second main pattern, as shown in the lower portion in FIG. 5B, are obtained. In addition to this, some murmur patterns are further obtained beside the main pattern. Apparently, a second pattern sample of the CWT data, as best shown in two arrows in FIG. 5B, indicates the heart of second patient suffering from diseases of continuous murmur and patent ductus arteriosum (PDA).

TABLE 1
Characteristics of CWT data of first and second patients processed
by the heart sound processing method of the present invention

| | slight AR | |
|---|---|---|
| diagnosis of disease | & slight AS | PDA |
| band of first maximum | 16 | 26 |
| amplitude (Hz) | | |
| first main frequency (Hz) | 27 | 26 |
| band of second maximum | 12 | 30 |
| amplitude (Hz) | | |
| second main frequency (Hz) | 17 | 28 |
| time interval of two maximum | 0.074 | 0.074 |
| amp. points (Sec.) | | |

Referring again to FIGS. 1 and 2, the heart sound processing method in accordance with the preferred embodiment of the present invention includes the step S3: automatically or semi-automatically converting the heart sound data fragments with short-time Fourier transformation by the heart sound processing unit 20 to obtain STFT data which can be shown as a second cardiopathy pattern.

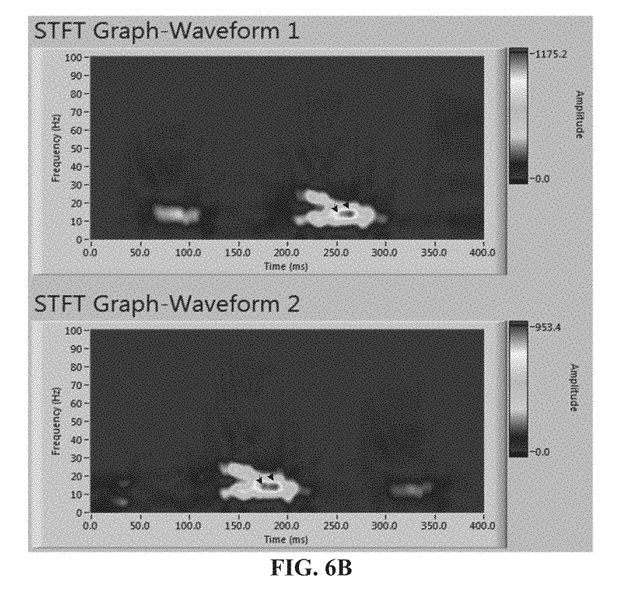

FIG. 6A shows an image of STFT data (two fragments) of slight aortic regurgitation and slight aortic stenosis collected from the first patient and processed by the heart sound processing method and system for detecting cardiopathy in accordance with the preferred embodiment of the present invention, corresponding to those shown in FIG. 5A. Referring to FIG. 6A, after STFT processed, a first STFT pattern, as shown in the upper portion in FIG. 6A, and a second STFT pattern, as shown in the lower portion in FIG. 6A, are obtained to compare the STFT data. Apparently, a first pattern sample of the STFT data has a first maximum amplitude and a second maximum amplitude, as best shown in two arrows in FIG. 6A, which also indicate the heart of first patient suffering from diseases of slight aortic regurgitation and slight aortic stenosis.

Next, FIG. 6B shows an image of STFT data (two fragments) of continuous murmur and patent ductus arteriosum collected from the second patient and processed by the heart sound processing method and system for detecting cardiopathy in accordance with the preferred embodiment of the present invention, corresponding to those shown in FIG. 5B. Referring to FIG. 6B, after STFT processed, a first wider STFT pattern, as shown in the upper portion in FIG. 6B, and a second wider STFT pattern, as shown in the lower portion in FIG. 6B, are obtained. In addition to this, some murmur patterns (i.e. narrower STFT patterns) are further obtained beside the larger STFT pattern. Apparently, a second pattern sample of the STFT data, as best shown in two arrows in FIG. 6B, indicates the heart of second patient suffering from diseases of continuous murmur and patent ductus arteriosum.

TABLE 2
Characteristics of STFT data of first and second patients processed
by the heart sound processing method of the present invention

| | slight AR | |
|---|---|---|
| diagnosis of disease | & slight AS | PDA |
| band of first maximum | 8 | 6 |
| amplitude (Hz) | | |
| first main frequency (Hz) | 14 | 14 |
| band of second maximum | 9 | 5 |
| amplitude (Hz) | | |
| second main frequency (Hz) | 13 | 14 |
| time interval of two maximum | 0.119 | 0.125 |
| amp. points (Sec.) | | |

Referring again to FIGS. 1 and 2, the heart sound processing method in accordance with the preferred embodiment of the present invention includes the step S3: automatically or semi-automatically calculating the CWT data and the STFT data to seek at least one correlation between time and frequency by the heart sound processing unit 20, or comparing the CWT data and the STFT data with ultrasound data samples of cardiopathy and further seeking the correlation between time and frequency for identifying cardiopathy. In a preferred embodiment, the at least one correlation is applied to identify cardiopathy from CWT data and STFT data of heart sound data collected from another patient to generate a predictable result of correlation coefficients which includes a disease of ventricular septal defect (VSD) or atrial septal defect (ASD).

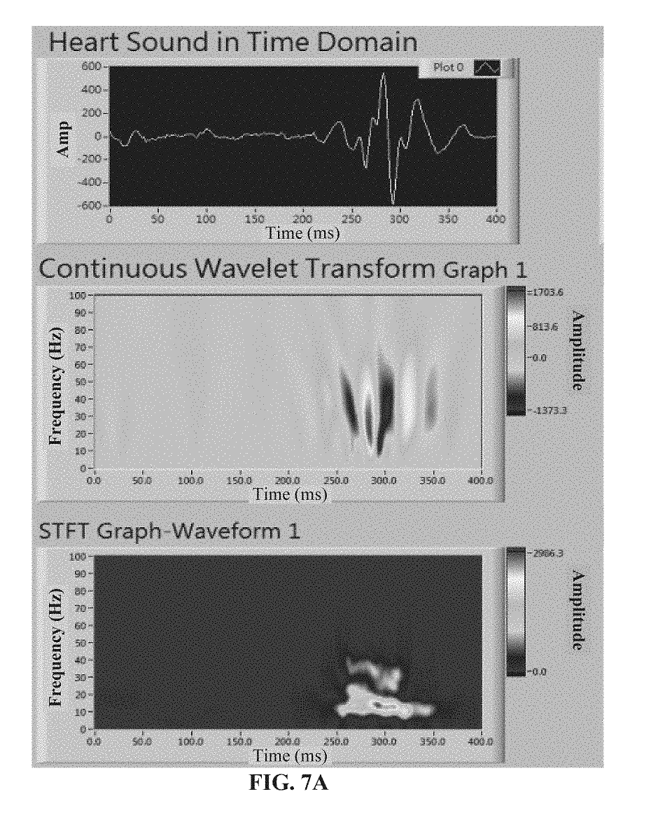

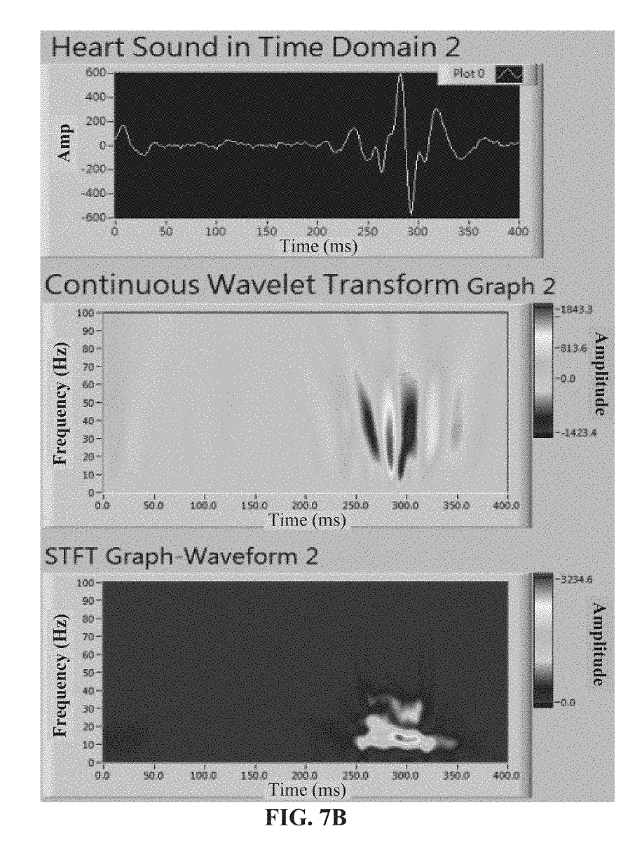

FIG. 7A shows a series of images of first original heart sound data fragment collected from the first patient to compare with first CWT data and STFT data processed by the heart sound processing method and system for detecting cardiopathy in accordance with the preferred embodiment of the present invention. FIG. 7B shows a series of images of second original heart sound data fragment collected from to compare with second CWT data and STFT data the first patient processed by the heart sound processing method and system for detecting cardiopathy in accordance with the preferred embodiment of the present invention. Referring again to FIGS. 7A and 7B, in pattern-comparing operation for the first patient, the second original heart sound data fragment, the second CWT data and the second STFT data, as shown in FIG. 7B are selectively and suitably adjusted to correspond to the first original heart sound data fragment, the first CWT data and the first STFT data, as shown in FIG. 7A, to thereby calculate correlations of a first pattern, as shown in TABLE 3.

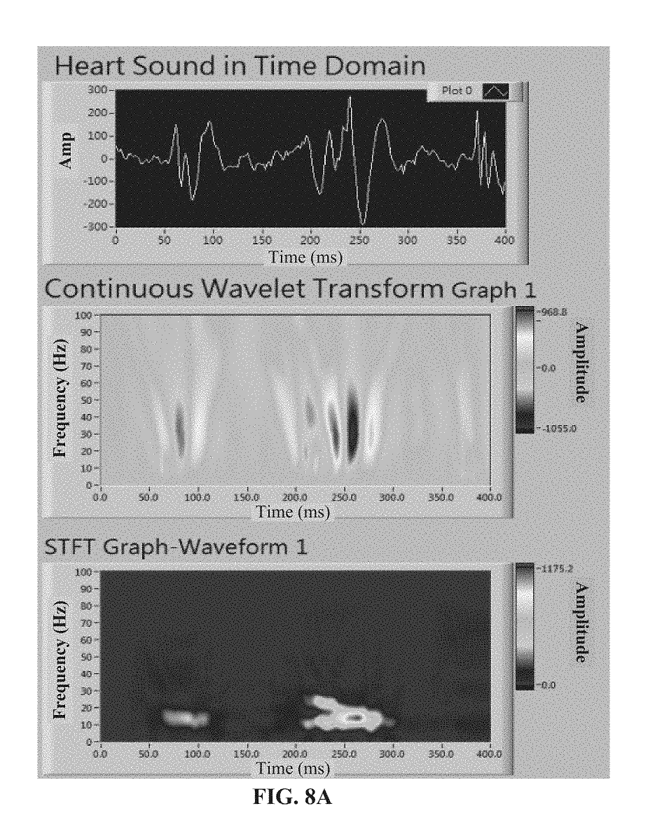

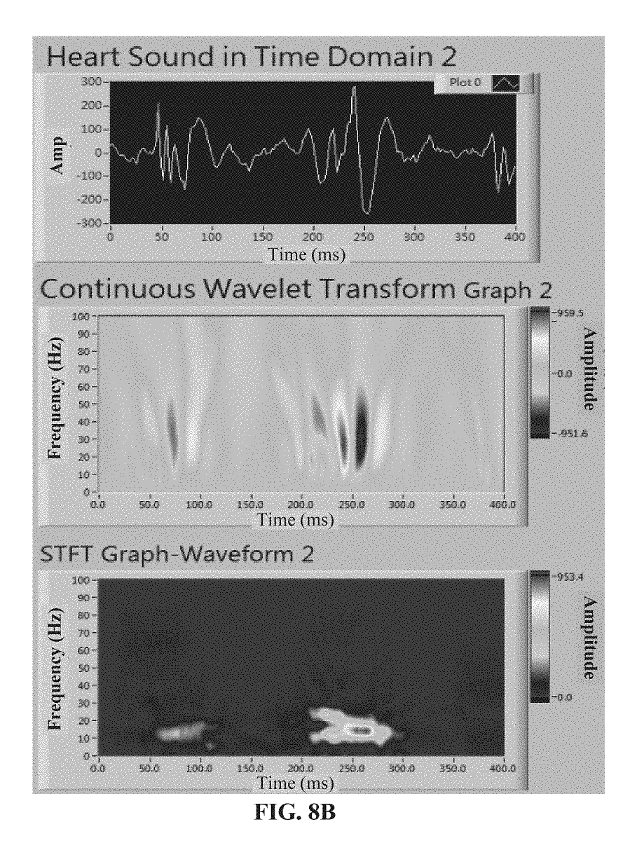

FIG. 8A shows a series of images of first original heart sound data fragment collected from the second patient to compare with first CWT data and STFT data processed by the heart sound processing method and system for detecting cardiopathy in accordance with the preferred embodiment of the present invention. FIG. 8B shows a series of images of second original heart sound data fragment collected from the second patient to compare with second CWT data and STFT data processed by the heart sound processing method and system for detecting cardiopathy in accordance with the preferred embodiment of the present invention. Referring again to FIGS. 8A and 8B, in another pattern-comparing operation for the second patient, the second original heart sound data fragment, the second CWT data and the second STFT data, as shown in FIG. 8B are selectively and suitably adjusted to correspond to the first original heart sound data fragment, the first CWT data and the first STFT data, as shown in FIG. 8A, to thereby calculate correlations of a second pattern, as shown in TABLE 3.

TABLE 3
Correlations of heart sound data of first and second
patients processed by the heart sound processing
method of the present invention

| | First | Second | Correl. | Correl. |
|---|---|---|---|---|
| Patient | main freq. | main freq. | of CWT | of STFT |
| first | 14 Hz | 13 Hz | r = 99.45% | r = 94.32% |
| second | 14 Hz | 14 Hz | r = 78.88% | r = 98.86% |

In a preferred embodiment, the first CWT data and the first STFT data are calculated with Pearson product-moment coefficient or other suitable coefficient. The Pearson product-moment coefficient applied in the present invention is in the form

$$r = \frac{\sum {z}_{x} {z}_{y}}{n}$$

where zₓ and z_{y} are standardized values z of x and y, and r is coefficient of correlation.

Although the invention has been described in detail with reference to its presently preferred embodiment, it will be understood by one of ordinary skills in the art that various modifications can be made without departing from the spirit and the scope of the invention, as set forth in the appended claims.

What is claimed is:

1. A heart sound processing method for detecting cardiopathy comprising:
partitioning first heart sound data to obtain a plurality of first heart sound data fragments;
converting the first heart sound data fragments with continuous wavelet transformation to obtain first CWT data;
converting the first heart sound data fragments with short-time Fourier transformation to obtain first STFT data; and
comparing the first CWT data and the first STFT data with at least one ultrasound data sample of cardiopathy and further seeking at least one correlation between time and frequency for identifying cardiopathy.

2. The method as defined in claim 1, wherein the at least one correlation is applied to identify cardiopathy from second CWT data and second STFT data of second heart sound data collected from a patient to generate a predictable result of correlation coefficients.

3. The method as defined in claim 2, wherein the predictable result of correlation coefficients includes a disease of ventricular septal defect or atrial septal defect.

4. The method as defined in claim 1, wherein the first CWT data and the first STFT data are compared with the ultrasound data to seek a maximum frequency point of heart sound, a maximum amplitude point of heart sound and at least one time interval of two maximum frequency points or two maximum amplitude points.

5. The method as defined in claim 1, wherein the first CWT data and the first STFT data are calculated with Pearson product-moment coefficient.

6. The method as defined in claim 1, wherein the first heart sound data is compared with ECG data for identifying cardiopathy.

7. A heart sound processing system for detecting cardiopathy comprising:
a heart sound receiving unit provided to receive heart sound data;
a heart sound processing unit connected with the heart sound receiving unit, with partitioning the heart sound data to obtain a plurality of heart sound data fragments, with converting the heart sound data fragments with continuous wavelet transformation to obtain CWT data, with converting the heart sound data fragments with short-time Fourier transformation to obtain STFT data;
a data storage unit connected with the heart sound processing unit, with the data storage unit storing at least one set of ultrasound data samples of cardiopathy; and
an output unit connected with the heart sound processing unit, with the output unit outputting a predictable result of correlation coefficients;
wherein the first CWT data and the first STFT data are compared with the at least one set of ultrasound data samples of cardiopathy and are further calculated to seek at least one correlation between time and frequency for identifying cardiopathy.

8. The system as defined in claim 7, wherein the heart sound receiving unit includes a first receiver unit and a second receiver unit to attach to a first predetermined position and a second predetermined position for synchronously collecting different heart sound data.

9. The system as defined in claim 7, wherein the heart sound receiving unit is configured to attach to a predetermined position of human skin.

10. The system as defined in claim 7, wherein heart sounds of the heart sound data fragments have a range of frequencies between 1 Hz and 100 Hz.

11. The system as defined in claim 7, wherein each of the heart sound data fragments includes a predetermined amount of continuous heart sound signals.

12. The system as defined in claim 7, wherein a pathologic murmur signal is detected in the heart sound data fragment to identify cardiopathy.

13. The system as defined in claim 7, wherein the pathologic murmur signal includes a systolic heart murmur signal or a diastolic heart murmur signal.

14. A heart sound processing method for detecting cardiopathy comprising:
partitioning first heart sound data to obtain a plurality of first heart sound data fragments;
converting the first heart sound data fragments with continuous wavelet transformation to obtain first CWT data;
converting the first heart sound data fragments with short-time Fourier transformation to obtain first STFT data; and
calculating the first CWT data or the first STFT data to seek at least one correlation between time and frequency for identifying cardiopathy.

15. The method as defined in claim 14, wherein the at least one correlation is applied to identify cardiopathy from second CWT data and second STFT data of second heart sound data collected from a patient to generate a predictable result of correlation coefficients.

16. The method as defined in claim 15, wherein the predictable result of correlation coefficients includes a disease of ventricular septal defect or atrial septal defect.

17. The method as defined in claim 14, wherein the first CWT data and the first STFT data are compared with the ultrasound data to seek a maximum frequency point of heart sound, a maximum amplitude point of heart sound and at least one time interval of two maximum frequency points or two maximum amplitude points.

18. The method as defined in claim 14, wherein the first CWT data and the first STFT data are calculated with Pearson product-moment coefficient.

19. The method as defined in claim 14, wherein the first heart sound data is compared with ECG data for identifying cardiopathy.
